(19) United States
(12) Patent Application Publication
(10) Pub. No.: US 2026/0157961 A1
(43) Pub. Date: Jun. 11, 2026

(54) APPLICATION OF NANOGEL IN USE OR PREPARATION OF ANTI-TUMOR DRUG

(71) Applicant: HUAZHONG UNIVERSITY OF SCIENCE AND TECHNOLOGY, Hubei (CN)

(72) Inventors: Zifu Li, Hubei (CN); Xiangliang YANG, Hubei (CN)

(73) Assignee: HUAZHONG UNIVERSITY OF SCIENCE AND TECHNOLOGY, Hubei (CN)

(21) Appl. No.: 19/389,053

(22) Filed: Nov. 14, 2025

(30) Foreign Application Priority Data
Dec. 11, 2024 (CN) 202411821110.4

(51) Int. Cl. A61K 9/06; A61K 9/00; A61K 31/785; A61P 35/00

(52) U.S. Cl. CPC A61K 9/06; A61K 9/0019; A61K 31/785; A61P 35/00

(57) ABSTRACT

The disclosure belongs to the technical field of nanogel preparation, and more specifically relates to an application of a nanogel in use or preparation of an anti-tumor drug. The experiments of the disclosure found that the nanogel with appropriate hardness can promote the expression of M1-related proteins CD86 and iNOS, upregulate the expression of CD86 mRNA and iNOS mRNA, and reverse polarize M2 tumor-associated macrophages to M1. Further studies found that after using the nanogel to reverse polarize M2 tumor-associated macrophages, proliferation of tumor cells can be inhibited, apoptosis of tumor cells can be induced, and tumor cells can be phagocytized. In addition, directly delivering the nanogel with appropriate hardness into a tumor through intratumoral injection can effectively inhibit tumor growth.

### CROSS-REFERENCE TO RELATED APPLICATION

This application claims the priority benefit of China application serial no. 202411821110.4, filed on Dec. 11, 2024. The entirety of the above-mentioned patent application is hereby incorporated by reference herein and made a part of this specification.

### BACKGROUND

### Technical Field

The disclosure belongs to the technical field of nanogel preparation, and more specifically relates to an application of a nanogel in use or preparation of an anti-tumor drug.

### Description of Related Art

Macrophages are important participants in maintaining homeostasis and regulating immune responses in healthy tissues. Their main function is to balance the activation of the inflammatory cascade in the context of tissue damage, while initiating tissue repair in parallel with identification of homeostasis disruptions caused by harmful organisms, apoptotic cells, cell debris, or toxic metabolic byproducts. Tumor cell transformation triggers macrophage polarization to restore the balance within the tumor microenvironment (TME). However, because growing tumors never reach steady-state equilibrium, the TAM phenotype remains locked in a cycle that ultimately favors immune cell activation and tissue repair. Exploring macrophage-intrinsic features selected by tumor cells is crucial for predicting their contributions to cancer progression and determining therapeutically exploitable vulnerabilities.

As the most widely distributed immune cells in the tumor microenvironment, tumor-associated macrophages (TAMs) account for about 50%. TAMs play an important role in tumor growth, invasion, and metastasis. There are two main subtypes of TAMs: the pro-inflammatory and tumoricidal M1-like phenotype and the anti-inflammatory and pro-repair M2-like phenotype. The plasticity of tumor-associated macrophages decides that the phenotype and the function may be switched in response to environmental stimuli. Therefore, TAMs are considered a potential anti-cancer target. Common strategies for using nanomedicines to target TAMs for anti-tumor objectives include macrophage polarization reprogramming, regulating the related tumor immune microenvironment, etc.

The literature (ACS Nano. 2019, 13, 12671-12686) uses nanoparticle encapsulation (NE) as a loading system to provide an immunotherapy platform of freeze-dried NEs loaded with TLR7/8a, which can induce T cell activation and stimulation enhancement and macrophage polarization, reprogram M2 TAMs in tumor tissues into M1 TAMs, and transform tumor cells from cold tumors into hot tumors, which can better respond to external drug stimulation, thereby improving the anti-tumor effect.

Currently, there are no reports on the effect of nanogels with different hardness on the reverse polarization of M2 tumor-associated macrophages and tumor growth.

### SUMMARY

In response to the defects of the prior art, the objective of the disclosure is to provide an application of a nanogel in use or preparation of an anti-tumor drug. Reverse polarization of M2 tumor-associated macrophages to M1 through synthesizing the nanogel with appropriate hardness effectively inhibits the tumor cell growth and improves the anti-tumor effect.

To achieve the above objective, in an aspect, the disclosure provides an application of a nanogel in use or preparation of an M2 TAMs reverse polarization drug. A Young's modulus of the nanogel is 20 to 600 KPa to reverse polarize M2 TAMs to M1.

In a second aspect, the disclosure also provides an application of a nanogel in use or preparation of an anti-tumor drug. An active ingredient of the anti-tumor drug is the nanogel. A Young's modulus of the nanogel is 20 to 600 KPa. The nanogel reverse polarizes M2 TAMs to M1 when used for anti-tumor treatment, thereby inhibiting proliferation of tumor cells.

Preferably, the Young's modulus of the nanogel is 50 to 600 KPa.

Preferably, the nanogel is obtained by a polymerization reaction of a monomer initiated through an initiator in an aqueous phase under a condition of presence of a cross-linking agent and a surfactant.

The Young's modulus of the nanogel is regulated through regulating a molar ratio of the cross-linking agent to the monomer.

Preferably, the monomer includes one or more of a temperature-responsive monomer, a pH-responsive monomer, and a reduction-responsive monomer.

Preferably, the temperature-responsive monomer is one or more of N-isopropylmethacrylamide, N-isopropylacrylamide, and N-ethylacrylamide.

Preferably, the pH-responsive monomer is one or more of methacrylic acid, acrylic acid, and 2-acrylamido-2-methyl-1-propanesulfonic acid.

Preferably, the cross-linking agent is one or more of N,N′-bis(acryloyl)cystamine, N,N′-methylenebisacrylamide, and N,N′-vinylbisacrylamide.

Preferably, the initiator is one or more of potassium persulfate, sodium persulfate, and tert-butyl hydroperoxide.

Preferably, the surfactant is one or more of sodium lauryl sulfate, sodium lauryl sulfonate, and lecithin.

Preferably, the monomer includes a temperature-responsive monomer and a pH-responsive monomer. A molar ratio of the pH-responsive monomer to the temperature-responsive monomer is (3˜8):100.

Preferably, a molar ratio of the cross-linking agent to the temperature-responsive monomer is (1˜20):100.

Preferably, a mass ratio of the initiator to the temperature-responsive monomer is (5˜15):550.

Preferably, a mass ratio of the surfactant to the temperature-responsive monomer is (20˜35):550.

Preferably, a reaction temperature of the polymerization reaction is 70° C. to 85° C., and a reaction time is 4 to 8 hours.

Preferably, the tumor cells include one or more of liver cancer cells, breast cancer cells, colon cancer cells, lung cancer cells, esophageal squamous cell cancer cells, gastric cancer cells, ovarian cancer cells, prostate cancer cells, pancreatic cancer cells, lymphoma cells, melanoma cells, and glioblastoma cells.

Preferably, when the nanogel is used for anti-tumor treatment, the nanogel is administered through intratumoral injection.

Preferably, a dosage of the nanogel is 1 to 10 μg/mm³.

In general, the above technical solutions conceived by the disclosure have the following technical advantages compared with the prior art.

(1) The disclosure provides the application of the nanogel in use or preparation of the M2 TAMs reverse polarization drug (that is, a drug that reverse polarizes M2 tumor-associated macrophages to M1). Experiments found that the nanogel with appropriate hardness can promote the expression of M1-related proteins CD86 and iNOS, upregulate the expression of CD86 mRNA and iNOS mRNA, and reverse polarize M2 tumor-associated macrophages to M1. Further studies found that the greater the hardness of the nanogel, the better the effect of reverse polarization of M2 tumor-associated macrophages to M1.

(2) The disclosure provides the application of the nanogel in use or preparation of the anti-tumor drug. Experiments found that after using the nanogel with appropriate hardness to reverse polarize M2 tumor-associated macrophages, proliferation of the tumor cells can be inhibited, apoptosis of the tumor cells can be induced, and the tumor cells can be phagocytized. In addition, directly delivering the nanogel with appropriate hardness into the tumor through intratumoral injection can inhibit tumor growth, and the greater the hardness of the nanogel, the better the effect of inhibiting tumor growth. The disclosure implements the optimal anti-tumor treatment strategy through fully utilizing the mechanical properties of the nanogel and has great potential for clinical transformation and application.

### BRIEF DESCRIPTION OF THE DRAWINGS

FIG. 1 shows a diameter distribution and surface charge of a nanogel provided in an embodiment of the disclosure, wherein content A is the diameter distribution and content B is the surface charge.

FIG. 2 shows a transmission electron microscope image of the nanogel provided in an embodiment of the disclosure.

FIG. 3 shows an atomic force microscope image and Young's modulus of the nanogel provided in an embodiment of the disclosure, wherein content A is the atomic force microscope image and content B is the Young's modulus.

FIG. 4 shows responsiveness of the nanogel provided in an embodiment of the disclosure, wherein content A is temperature responsiveness, content B is pH responsiveness, and content C is reduction responsiveness.

FIG. 5 shows an effect of the nanogel provided in an embodiment of the disclosure on reverse polarization of M2 macrophages, wherein content A is an expression of an M1-related protein, content B is an mRNA expression level, and content C is a CD86 relative fluorescence intensity.

FIG. 6 shows cell viability of tumor cells after reverse polarization of M2 macrophages and after co-incubation with the tumor cells by the nanogel provided in an embodiment of the disclosure, wherein content A is H22 cells and content B is 4T1 cells.

FIG. 7 shows an apoptosis rate of tumor cells after reverse polarization of M2 macrophages and after co-incubation with the tumor cells by the nanogel provided in an embodiment of the disclosure, wherein content A is H22 cells and content B is 4T1 cells.

FIG. 8 shows a phagocytic ability of tumor cells after reverse polarization of M2 macrophages and after co-incubation with the tumor cells by the nanogel provided in an embodiment of the disclosure, wherein content A is a cell population selected on a flow cytometer according to different fluorescently labeled cells and content B is a phagocytic ratio.

FIG. 9 shows a killing effect of a mouse tumor after reverse polarization of M2 macrophages and co-incubation with tumor cells by the nanogel provided in an embodiment of the disclosure, wherein content A is an experimental process, content B is a tumor volume, content C is a tumor weight, and content D is a tumor image.

FIG. 10 shows a degree of reverse polarization of M2 macrophages after reverse polarization of M2 macrophages and after co-incubation with tumor cells by the nanogel provided in an example of the disclosure, wherein content A is an immunofluorescence intensity and content B is a CD86⁺ positive rate.

FIG. 11 shows an experimental process of intratumoral injection of the nanogel provided in an embodiment of the disclosure into a mouse with breast cancer.

FIG. 12 shows an inhibitory effect on mouse tumor growth after intratumoral injection of the nanogel provided in an embodiment of the disclosure in a mouse with breast cancer.

FIG. 13 shows an effect of a relative growth rate on a mouse tumor after intratumoral injection of the nanogel provided in an embodiment of the disclosure in a mouse with breast cancer.

### DESCRIPTION OF THE EMBODIMENTS

In order for the objectives, the technical solutions, and the advantages of the disclosure to be clearer, the disclosure is further described in detail below with reference to the drawings and the embodiments. It should be understood that the specific embodiments described herein are only used to explain the disclosure and are not intended to limit the disclosure.

In the description of the disclosure, it should be understood that the term “and/or” is a description of an association relationship between associated objects, indicating that three relationships may be present. For example, A and/or B may represent three situations where A is present alone, A and B are present at the same time, and B is present alone. In the disclosure, the symbol “/” represents that the associated objects are in an or relationship. For example, A/B represents A or B.

In the description of the embodiments of the disclosure, words such as “exemplary” or “for example” are used to indicate examples, illustrations, or descriptions. Any embodiment or design solution described as “exemplary” or “for example” in the embodiments of the disclosure should not be construed as being preferred or advantageous over other embodiments or design solutions. Rather, the use of words such as “exemplary” or “for example” are intended to present the related concepts in a specific manner.

In the description of the embodiments of the disclosure, unless otherwise specified, “multiple” means two or more.

The term “M2 TAMs reverse polarization drug” refers to a drug that may reverse polarize M2 tumor-associated macrophages (TAMs) to M1.

The term “intratumoral injection” or “intratumoral therapy” refers to a form of tumor treatment in which a therapeutically active ingredient is directly delivered into a tumor. Such manner of administration prevents systemic distribution and plasma and tissue clearance effects of intravenous administration and can more effectively implement targeted drug delivery and tumor treatment, the drug concentration at the lesion site is high, and the systemic drug distribution is low, which can effectively implement toxicity reduction and efficacy enhancement.

Nanogel is a nanohydrogel that has the advantages of a hydrogel and nanoparticles. Patent Document CN 115960305 A obtains a soft nanomedicine (the Young's modulus of the soft nanogel is 20 to 150 KPa) and a hard nanomedicine through regulating the cross-linking degree of the nanogel and using nanogels with different hardness as carriers to load an anti-tumor chemotherapy drug through electrostatic adsorption. Then, the nanomedicines are administered through intravenous injection. Experiments found that the soft nanomedicine can be enriched in the tumor site rather than the liver. At the same time, the ability of the soft nanomedicine to penetrate deep into the tumor from tumor blood vessels and the anti-tumor effect are significantly better than those of the hard nanogel. Unlike the prior art, the inventors of the disclosure accidentally discovered during the experiment that directly administering the nanogel with appropriate hardness into a mouse tumor through intratumoral injection can reverse polarize M2 tumor-associated macrophages to M1, effectively inhibiting tumor growth, and the anti-tumor effect is even better than the soft nanomedicine administered through intravenous injection. Based on this, the disclosure provides an application of a nanogel in use or preparation of an M2 TAMs reverse polarization drug. The Young's modulus of the nanogel is 20 to 600 KPa to reverse polarize M2 TAMs to M1.

The disclosure also provides an application of a nanogel in use or preparation of an anti-tumor drug. The active ingredient of the anti-tumor drug is the nanogel. The Young's modulus of the nanogel is 20 to 600 KPa. The nanogel can reverse polarize M2 tumor-associated macrophages to M1 when used for anti-tumor treatment, thereby inhibiting proliferation of tumor cells, inducing apoptosis of the tumor cells, phagocytizing the tumor cells, and implementing the anti-tumor effect.

The inventors unexpectedly found that after co-incubating the nanogel with appropriate hardness with M2 macrophages, the expression of M1-related proteins CD86 and iNOS can be promoted, the expression of CD86 mRNA and iNOS mRNA can be upregulated, and M2 tumor-associated macrophages can be reverse polarized to M1. Further studies found that the greater the hardness of the nanogel, the better the effect of reverse polarization of M2 tumor-associated macrophages to M1. Furthermore, directly delivering the nanogel with appropriate hardness into tumor through intratumoral injection can inhibit tumor growth, and the greater the hardness of the nanogel, the better the effect of inhibiting tumor growth.

In some embodiments, the Young's modulus of the nanogel is between 50 and 600 KPa, more preferably between 70 and 600 KPa.

In some embodiments, the nanogel is obtained by a polymerization reaction of a monomer initiated through an initiator in an aqueous phase under a condition of presence of a cross-linking agent and a surfactant, wherein the Young's modulus of the nanogel can be regulated through adjusting a molar ratio of the cross-linking agent to the monomer.

Synthesizing of the nanogel of the disclosure may adopt a common monomer for preparing a biocompatible nanogel. Different monomers may be used to give the nanogel different functions, such as hydrophilicity, pH responsiveness, and reduction responsiveness. The monomer includes, but is not limited to, one or more of a temperature-responsive monomer, a pH-responsive monomer, and a reduction-responsive monomer. In some embodiments, the temperature-responsive monomer is one or more of N-isopropylmethacrylamide, N-isopropylacrylamide, and N-ethylacrylamide. In some embodiments, the pH-responsive monomer is one or more of methacrylic acid, acrylic acid, and 2-acrylamido-2-methyl-1-propanesulfonic acid.

In a preferred embodiment, the monomer includes a temperature-responsive monomer and a pH-responsive monomer. More preferably, the temperature-responsive monomer is N-isopropylmethacrylamide, the pH-responsive monomer is methacrylic acid, and a molar ratio of the pH-responsive monomer to the temperature-responsive monomer is (3˜8):100.

In some embodiments, the cross-linking agent is one or more of N,N′-bis(acryloyl)cystamine, N,N′-methylenebisacrylamide, and N,N′-vinylbisacrylamide. The cross-linking agent is more preferably a reduction-responsive cross-linking agent. In a preferred embodiment, the cross-linking agent is N,N′-bis(acryloyl)cystamine.

In some embodiments, a molar ratio of the cross-linking agent to the temperature-responsive monomer is (1˜20):100, and preferably (2˜20):100. The greater the molar ratio, the greater the Young's modulus of the prepared nanogel correspondingly, that is, the greater the hardness.

In some embodiments, the initiator is one or more of potassium persulfate, sodium persulfate, and tert-butyl hydroperoxide.

In some embodiments, a mass ratio of the initiator to the temperature-responsive monomer is (5˜15):550.

In some embodiments, the surfactant is one or more of sodium lauryl sulfate, sodium lauryl sulfonate, and lecithin.

In some embodiments, a mass ratio of the surfactant to the temperature-responsive monomer is (20˜35):550.

In a preferred embodiment of the disclosure, the cross-linking degree of the nanogel is regulated through adjusting the molar ratio of the cross-linking agent to the temperature-responsive monomer, thereby regulating the hardness of the nanogel. The greater the molar ratio of the two, the greater the hardness of the prepared nanogel. In addition, the hydrodynamic diameter of the nanogel may be regulated through adjusting the amount of surfactant used, so that the nanogel can be applied to the tumor site through intratumoral injection. In some embodiments, the average hydrodynamic diameter of the nanogel in a PBS solution at 37° C. is 150 to 300 nm. The Young's modulus of the nanogel is 79.0 to 439.2 KPa.

In some embodiments, a reaction temperature of the polymerization reaction is 70° C. to 85° C., and a reaction time is 4 to 8 hours.

In some embodiments, the tumor cells include, but are not limited to, one or more of liver cancer cells, breast cancer cells, colon cancer cells, lung cancer cells, esophageal squamous cell cancer cells, gastric cancer cells, ovarian cancer cells, prostate cancer cells, pancreatic cancer cells, lymphoma cells, melanoma cells, and glioblastoma cells.

In some embodiments, when the nanogel is used for anti-tumor treatment, the nanogel is administered through intratumoral injection.

In some embodiments, a dosage of the nanogel is 1 to 10 μg/mm³.

In actual application, persons skilled in the art may perform intratumoral injection again 4 to 10 days after the first intratumoral injection according to actual situations. The objective is to enhance the anti-tumor effect.

It should be understood that materials with the same or similar types, models, qualities, properties, or functions as those of reagents and instruments used in the following examples may be used to implement the disclosure. Unless otherwise specified, the experimental methods used in the following examples are conventional methods. Unless otherwise specified, the materials and the reagents used in the following examples may be obtained from commercial sources.

When preparing the nanogel in the following examples of the disclosure, the molar ratio of the cross-linking agent to the temperature-responsive monomer is defined as X:100, the corresponding cross-linking degree of the nanogel is X%, and the prepared nanogel is expressed as X% NGs.

The following are examples.

Example 1 Effects of nanogels with different hardness on the reverse polarization of m2 macrophages

### 1.1 Preparation of Nanogels

550 mg of a temperature-responsive monomer N-isopropylmethylpropionamide and 35 mg of a surfactant sodium lauryl sulfate were ultrasonically dissolved in 80 mL of ultrapure water to obtain a mixed solution. 22.5 g of a cross-linking agent N,N′-bis(acryloyl)cystamine was ultrasonically dissolved in 0.5 mL of ethanol and then added to the mixed solution. 3.65 mL of a pH-responsive monomer methacrylic acid was dissolved in 6.35 mL of ultrapure water, and 50 μL was added to the solution. The obtained solution was evacuated for 10 minutes, argon was then introduced for three cycles to fully remove oxygen from the solution, then heated to 80° C. and maintained for 10 minutes. 10 mg of an initiator potassium persulfate was weighted, 0.5 mL of ultrapure water was added, ultrasonically dissolved, then added to the heated solution using a syringe to initiate a polymerization reaction, and the reaction time was 6 hours. After the reaction ended, the reaction solution was cooled to room temperature and transferred to an ultrafiltration tube with a cutoff value of 10 kDa, an unreacted monomer and other impurities were removed by centrifugation at 2000 rpm, while removing excess water, ultrapure water was repeatedly added to wash 3 times after concentration, and concentrated to obtain 8 mL of a concentrated solution. 300 μL of the concentrated solution was taken, dried, and weighed, and a solid content in the concentrated solution was calculated. According to the calculation result, the concentrated solution was diluted to 20 mg/mL to obtain a nanogel solution with a cross-linking degree of 2% (denoted as 2% NGs), which was stored at 4° C. for future use.

According to the above steps, nanogel solutions with cross-linking degrees of 5%, 10%, and 15% were respectively prepared, wherein amounts of the surfactant sodium lauryl sulfate used were respectively 30 mg, 25 mg, and 20 mg, amounts of the cross-linking agent N,N′-bis(acryloyl)cystamine used were respectively 56.3 mg, 112.6 mg, and 168.9 mg, and the cross-linking agents were respectively ultrasonically dissolved in 1 mL, 2 mL, and 3 mL of ethanol, and then added to the mixed solution. Finally, 5% NGs, 10% NGs, and 15% NGs were prepared and stored at 4° C. for future use.

### 1.2 Physical Properties of Nanogels

10 μL of the nanogel solution prepared in step 1.1 was taken and respectively dispersed in 1 mL of the PBS solution and 1 mL of ultrapure water, dynamic light scattering was used to detect the hydrodynamic diameter and zeta potential of the nanogel, a detection temperature was 37° C., and an equilibration time was 15 minutes.

The nanogel solution prepared in step 1.1 was taken and dispersed with ultrapure water to a concentration of 0.01 mg/mL, 10 μL of the dispersed solution was taken and added dropwise to a carbon support film, dried naturally, 10 μL of a 1% phosphotungstic acid aqueous solution was added dropwise to the carbon support film after drying, stained for 2 minutes, a filter paper was used to absorb the excess phosphotungstic acid solution along the edge of the carbon support film after staining, 10 μL of ultrapure water was added dropwise to wash for 1 minute, a filter paper was used to absorb the excess ultrapure water along the edge of the carbon support film after washing, and the morphology of the nanogel was observed using a transmission electron microscope after drying naturally.

A coverslip was soaked in 1% of a polyethyleneimine aqueous solution for 24 hours to modify the surface of the coverslip with positive charges, the nanogel solution prepared in step 1.1 was taken and dispersed with ultrapure water to a concentration of 0.01 mg/mL, 10 μL of the dispersed solution was added dropwise to the positively charged coverslip, which was electrostatically adsorbed for 10 minutes to remove the excess dispersed solution, 300 μL of ultrapure water was then added dropwise to wash away the unadsorbed nanogel, the height and the Young's modulus of the nanogel were detected using an atomic force microscope, and a detection environment was liquid phase, wherein image acquisition was in a contact mode, and detection of the Young's modulus was in a tapping mode.

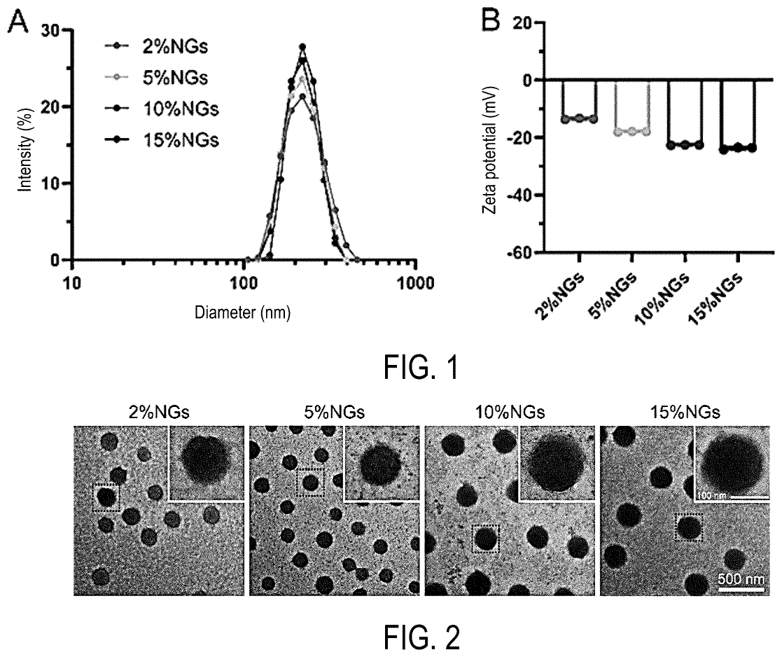

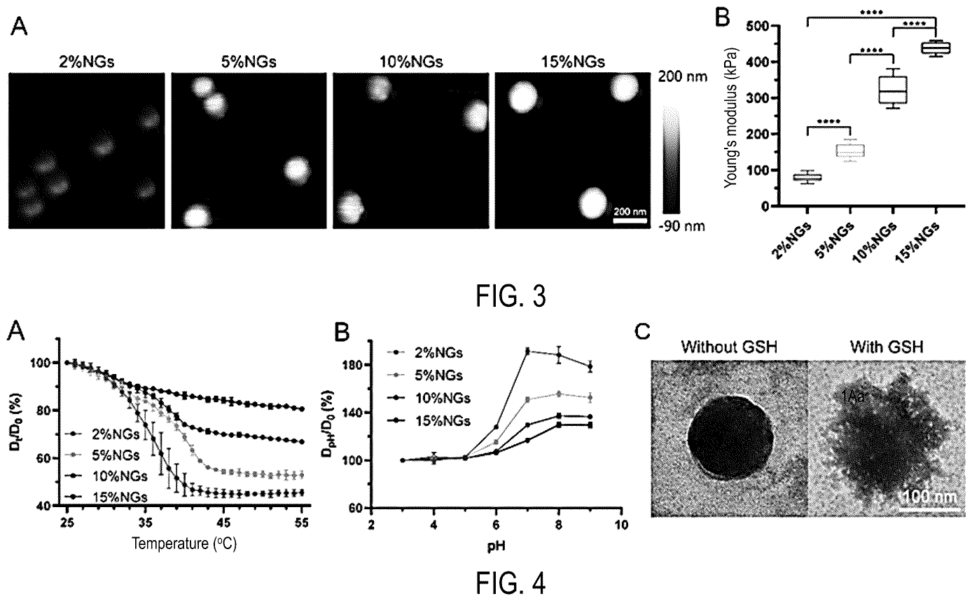

The results show that as can be seen from content A of FIG. 1 and content B of FIG. 1, the average hydrodynamic diameter of the nanogels with different hardness prepared in the example was about 220 nm, and the surface charge was all negative, which slightly increased with the increase in the cross-linking degree. As can be seen from FIG. 2, the nanogels with different hardness prepared in the example were all spherical with uniform diameter distributions. As can be seen from content A of FIG. 3 and content B of FIG. 3, the nanogels with different hardness prepared in the example were all spherical with uniform diameter distributions, and the Young's moduli were 79.0 to 439.2 kPa.

### 1.3 Triple Responsiveness of Nanogels

10 μL of the nanogel solution prepared in step 1.1 was taken and respectively dispersed in 1 mL of ultrapure water at different temperatures, dynamic light scattering was used to detect the temperature responsiveness of the nanogel, a detection temperature range was 25 to 55° C., a temperature interval was 1° C., and the equilibration time was 1 minute.

10 μL of the nanogel solution prepared in step 1.1 was taken and dispersed in 1 mL of ultrapure water, pH was regulated to 3 9, dynamic light scattering was used to detect the pH responsiveness of the nanogel, the detection temperature is 25° C., and the equilibration time was 15 minutes.

10 μL of the nanogel solution prepared in step 1.1 was taken and dispersed in 1 mL of ultrapure water containing or not containing 10 mM of glutathione (GSH) and incubated at room temperature for 24 hours. Then, 10 μL of the dispersed solution was taken and added dropwise to the carbon support film, dried naturally, 10 μL of a phosphotungstic acid aqueous solution with a mass fraction of 1% was added dropwise to the carbon support film after drying, stained for 2 minutes, a filter paper was used to absorb the excess phosphotungstic acid solution along the edge of the carbon support film after staining, 10 μL of ultrapure water was added dropwise to wash for 1 minute, a filter paper was used to absorb the excess ultrapure water along the edge of the carbon support film after washing, and the structure of the nanogel was observed using the transmission electron microscope after drying naturally.

The results show that as can be seen from content A of FIG. 4, content B of FIG. 4, and content C of FIG. 4, as the temperature increased, the hydrodynamic diameter of the nanogel prepared in the example gradually decreased, and the shrinkage degree decreased with increase in the cross-linking degree. As the pH increased, the hydrodynamic diameter of the nanogel prepared in the example gradually increased, and the swelling degree decreased with increase in the cross-linking degree. After incubation with glutathione for 24 hours, the structure prepared in the example was destroyed. That is, the nanogel prepared in the example had excellent temperature responsiveness, pH responsiveness, and reduction responsiveness.

### 1.4 Effect of Nanogel on Reverse Polarization of M 2 Macrophages

1) Induction to form M2 macrophages. RAW264.7 mouse macrophages were seeded at a concentration of 1×10⁶ cells/well in a 6-well plate. 2 mL of a culture medium obtained by mixing IL-4, IL-13, and a DMEM containing 10% serum was then added to each well, wherein the dose of IL-4 was 20 μg/mL and the dose of IL-13 was 20 μg/mL, incubated in an incubator at 37° C. and 5% CO₂ for 24 hours, and an upper layer of the culture medium was then aspirated to obtain M2 macrophages.

2) The nanogel solutions with different hardness prepared in step 1.1 were mixed with the DMEM containing 10% serum to obtain a mixed culture medium containing the nanogels with different hardness, wherein the concentration of the nanogel was 200 μg/mL. 2 mL of the mixed culture medium containing the nanogels with different hardness was added to each well of a 6-well plate containing M2 macrophages, the cells were collected after incubation in an incubator for 24 hours, washed 3 times with the PBS solution, the cells were digested with trypsin, and the cells were collected by centrifugation. The DMEM containing 10% serum without adding any nanogel solution was used as a control group, the mixed culture medium containing 2% NGs nanogel was used as a 2% NGs treatment group, and the culture medium containing 15% NGs nanogel was used as a 15% NGs treatment group.

Immunoblotting was used to detect the expression of M1 and M2 related proteins. RT-PCR was used to detect the expression levels of iNOS mRNA and CD86 mRNA in cells. The cells collected by centrifugation were stained using PE anti-mouse CD86 on ice protected from light for 20 minutes, the dose was 25 μg/μL, the cells were then washed 2 times with the PBS solution, collected by centrifugation and a fluorescence intensity was detected using a flow cytometer, the detection channel was PE, and the degree of polarization of M1 macrophages was calculated according to the fluorescence intensity.

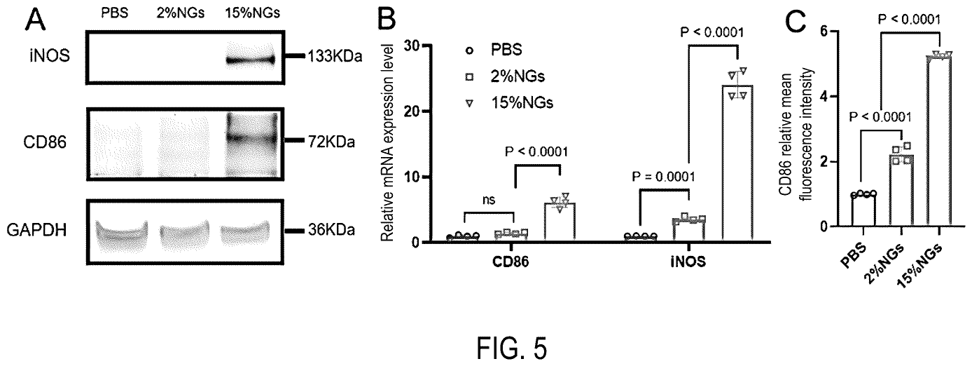

The results show that as can be seen from content A of FIG. 5, content B of FIG. 5, and content C of FIG. 5, compared with the control group, the 2% NGs treatment group and the 15% NGs treatment group could significantly upregulate the expression of M1-related proteins CD86 and iNOS, and the 15% NGs treatment group could significantly upregulate the expression of CD86 mRNA and iNOS mRNA. This indicates that the nanogels with different hardness can reverse polarize M2 macrophages to M1.

### Example 2: Killing of Tumor Cells After Reverse Polarization Treatment of M2 Macrophages by Nanogels

1) The method for induction to form M2 macrophages is the same as that of Example 1.

2) The nanogel solutions with different hardness prepared in step 1.1 were mixed with the DMEM containing 10% serum to obtain the mixed culture medium containing the nanogels with different hardness, wherein the concentration of the nanogel was 200 μg/mL. 2 mL of the mixed culture medium containing the nanogels with different hardness was added to each well of a 6-well plate containing M2 macrophages, incubated in an incubator for 24 hours, the upper layer of the culture medium was aspirated, 2 mL of the DMEM not containing serum was added to each well, incubated in an incubator at 37° C. and 5% CO₂ for 24 hours, and the upper layer of the culture medium was collected and set aside. The DMEM containing 10% serum without adding any nanogel solution was used as a M2 treatment group, the mixed culture medium containing PBS with an equal volume of nanogel was used as a PBS treatment group, the mixed culture medium containing 2% NGs nanogel was used as an M2+2% NGs treatment group, and the medium containing 15% NGs nanogel was used as an M2+15% NGs treatment group.

3) H22 cells were seeded at a concentration of 5×10³ cells/well in a 96-well plate, 200 μL of the upper layer of the culture medium was added to each well, incubated in an incubator at 37° C. and 5% CO₂ for 24 hours, 22 μL of CCK8 was then added to each well, incubated in an incubator at 37° C. and 5% CO₂ for 1 hour, light absorbance of the culture medium was measured at 450 nm using a microplate reader, and the results were processed and analyzed using Excel and Graphpad Prism to calculate the killing effect of a supernatant on H22 cells after treating M2 macrophages by the nanogels with different hardness.

4T1 cells were seeded at a concentration of 5×10³ cells/well in a 96-well plate, 200 μL of RPMI-1640 medium containing 10% serum was added to each well, incubated in an incubator at 37° C. and 5% CO₂ for 12 hours, after 4T1 cells adhered to a wall, the upper layer of the culture medium was aspirated, 200 μL of the upper layer of the culture medium was added to each well, incubated in an incubator at 37° C. and 5% CO₂ for 24 hours, and the upper layer of the culture medium was aspirated. Then, 100 μL of a culture medium obtained by mixing CCK8 and RPMI-1640 medium containing 10% serum according to a ratio of 1:9 was added to each well, incubated in an incubator at 37° C. and 5% CO₂ for 1 hour, the absorbance of the culture medium was measured at 450 nm using a microplate reader, and the results were processed and analyzed using Excel and Graphpad Prism to calculate the killing effect of the supernatant on 4T1 cells after treating M2 macrophages by the nanogels with different hardness.

The results show that as can be seen from content A of FIG. 6 and content B of FIG. 6, compared with other treatment groups, the viability of H22 cells and 4T1 cells in the 2% NGs treatment group and the 15% NGs treatment group was significantly decreased, showing an inhibitory effect on the proliferation of H22 cells and 4T1 cells. This indicates that the supernatant after the nanogel reverse polarized M2 macrophages to M1 can effectively kill the tumor cells.

### Example 3: Apoptosis of Tumor Cells After Reverse Polarization Treatment of M2 Macrophages by Nanogels

1) The method for induction to form M2 macrophages is the same as that of Example 1.

2) The nanogel solutions with different hardness prepared in step 1.1 were mixed with the DMEM containing 10% serum to obtain the mixed culture medium containing the nanogels with different hardness, wherein the concentration of the nanogel was 200 μg/mL. 2 mL of the mixed culture medium containing the nanogels with different hardness was added to each well of a 6-well plate containing M2 macrophages, incubated in an incubator for 24 hours, and the upper layer of the culture medium was aspirated. Then, 2 mL of the DMEM not containing serum was added to each well, incubated in an incubator at 37° C. and 5% CO₂ for 24 hours, and the upper layer of the culture medium was collected and set aside. The DMEM without adding the nanogel solution and containing only 10% serum was used as the control group, the mixed culture medium containing 2% NGs nanogel was used as the 2% NGs treatment group, and the culture medium containing 15% NGs nanogel was used as the 15% NGs treatment group.

3) H22 cells were seeded at a concentration of 1×10⁶ cells/well in a 6-well plate, 200 μL of the upper layer of the culture medium was added to each well, incubated in an incubator at 37° C. and 5% CO₂ for 24 hours, the upper layer of the culture medium was aspirated, collected by centrifugation, the cells were resuspended in 100 μL of PBS, 5 μL of Annexin V-FITC and 10 μL of PI staining solution were added, protected from light, reacted at room temperature for 15 minutes, washed 2 times with the PBS solution, collected by centrifugation and the fluorescence intensity was detected using a flow cytometer, the detection channels were PE and FITC, and the degree of apoptosis of H22 cells was calculated according to the fluorescence intensity.

4T1 cells were seeded at a concentration of 1×10⁶ cells/well in a 6-well plate, 200 μL of RPMI-1640 medium containing 10% serum was added to each well, incubated in an incubator at 37° C. and 5% CO₂ for 12 hours, after 4T1 cells adhered to a wall, the upper layer of the culture medium was aspirated, 200 μL of the upper layer of the culture medium was added to each well, incubated in an incubator at 37° C. and 5% CO₂ for 24 hours, the upper layer of the culture medium was aspirated, washed 3 times with the PBS solution, the cells were digested with trypsin, collected by centrifugation, the cells were resuspended in 100 μL of PBS, 5 μL of Annexin V-FITC and 10 μL of PI staining solution were added, protected from light, reacted at room temperature for 15 minutes, washed 2 times with the PBS solution, collected by centrifugation and the fluorescence intensity was detected using a flow cytometer, the detection channels were PE and FITC, and the degree of apoptosis of 4T1 cells was calculated according to the fluorescence intensity.

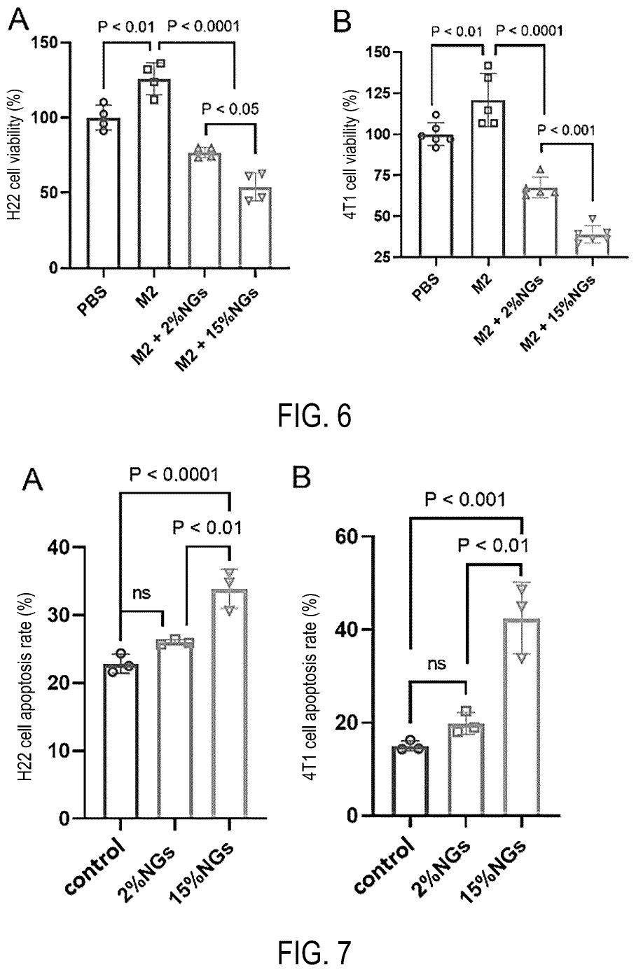

The results show that as can be seen from content A of FIG. 7 and content B of FIG. 7, compared with the control group, the apoptosis rates of H22 cells and 4T1 cells in the 2% NGs treatment group and the 15% NGs treatment group were increased, wherein the 15% NGs treatment group could effectively cause apoptosis of the tumor cells. This indicates that the supernatant after the nanogel reverse polarized M2 macrophages to M1 can effectively cause apoptosis of the tumor cells.

### Example 4 Phagocytosis of Tumor Cells After Reverse Polarization Treatment of M2 Macrophages by Nanogels

1) The method for induction to form M2 macrophages is the same as that of Example 1.

2) The nanogel solutions with different hardness prepared in step 1.1 were mixed with the DMEM containing 10% serum to obtain the mixed culture medium containing the nanogels with different hardness, wherein the concentration of the nanogel was 200 μg/mL. 2 mL of the mixed culture medium containing the nanogels with different hardness was added to each well of a 6-well plate containing M2 macrophages, incubated in an incubator for 24 hours, and the upper layer of the culture medium was aspirated. Then, 2 mL of the DMEM not containing serum was added to each well, incubated in an incubator at 37° C. and 5% CO₂ for 24 hours, and macrophages were collected. The DMEM containing 10% serum without adding the nanogel solution was used as the control group, the mixed culture medium containing 2% NGs nanogel was used as the 2% NGs treatment group, and the culture medium containing 15% NGs nanogel was used as the 15% NGs treatment group. The collected macrophages were washed 3 times with the PBS solution, digested with trypsin and then collected by centrifugation, then stained with FITC anti-mouse CD11b on ice protected from light for 20 minutes, and the dose was 25 μg/μL. The stained macrophages were seeded at a concentration of 1×10⁶ cells/well in a 6-well plate for later use.

3) 4T1 cells were seeded at a concentration of 1×10⁶ cells/well in a 6-well plate, 200 μL of RPMI-1640 medium containing 10% serum was added to each well, incubated in an incubator at 37° C. and 5% CO₂ for 24 hours, the upper layer of the culture medium was aspirated, 4T1 cells were collected and washed 3 times with the PBS solution, the cells were digested with trypsin and collected by centrifugation, then stained with PE anti-mouse EPCAM on ice protected from light for 20 minutes, and the dose was 25 μg/μL. The stained 4T1 cells were added to the 6-well plate containing the stained macrophages according to a concentration of 2×10⁶ cells/well, incubated in an incubator at 37° C. and 5% CO₂ for 4 hours, the upper layer of the culture medium was aspirated, the cells were collected and washed 3 times with the PBS solution, the cells were digested with trypsin and then collected by centrifugation, the fluorescence intensity of the cells was then detected using a flow cytometer, the detection channels were PE and FITC, and the degree of phagocytosis of 4T1 cells by macrophages was calculated according to the fluorescence intensity.

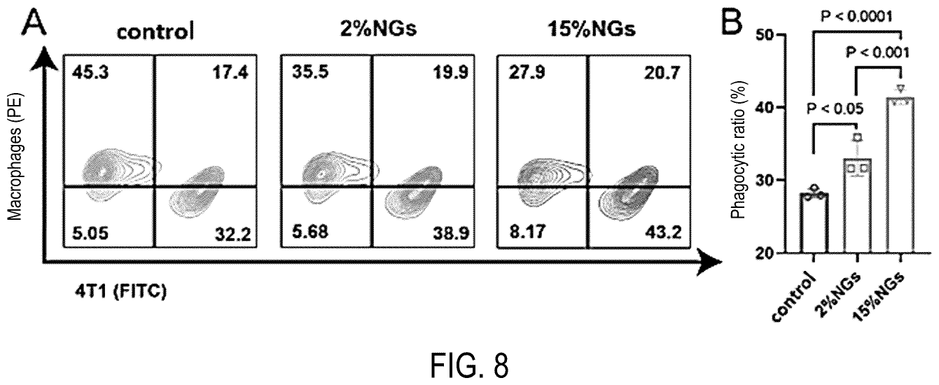

The results show that as can be seen from content A of FIG. 8 and content B of FIG. 8, compared with the control group, the ratios of 4T1 cells phagocytized by macrophages in the 2% NGs treatment group and the 15% NGs treatment group were significantly increased. This indicates that the nanogels with different hardness after reverse polarization treatment of M2 macrophages can effectively phagocytize the tumor cells.

### Example 5: Killing of Mouse Tumors After Reverse Polarization Treatment of M2 Macrophages by Nanogels

1) RAW264.7 mouse macrophages were seeded at a concentration of 1×10⁶ cells/well in a 6-well plate, and 2 mL of a culture medium obtained by mixing IL-4, IL-13, and DMEM containing 10% serum was added to each well, wherein the dose of IL-4 was 20 μg/mL and the dose of IL-13 was 20 μg/mL, incubated in an incubator at 37° C. and 5% CO₂ for 24 hours, and the upper layer of the culture medium was then aspirated to obtain M2 macrophages.

RAW264.7 mouse macrophages were seeded at a concentration of 1×10⁶ cells/well in a 6-well plate, 2 mL of the DMEM containing 10% serum was then added to each well, incubated in an incubator at 37° C. and 5% CO₂ for 24 hours, and the upper layer of the culture medium was then aspirated to obtain M0 macrophages.

RAW264.7 mouse macrophages were seeded at a concentration of 1×10⁶ cells/well in a 6-well plate, 2 mL of the DMEM containing 10% serum was then added to each well, incubated in an incubator at 37° C. and 5% CO₂ for 24 hours, the upper layer of the culture medium was aspirated, and 2 mL of a culture medium obtained by mixing LPS and the DMEM containing 10% serum was then added to each well, wherein the dose of LPS was 1 μg/mL, incubated in an incubator at 37° C. and 5% CO₂ for 24 hours, and the upper layer of the culture medium was then aspirated to obtain M1 macrophages.

3) The nanogel solutions with different hardness prepared in step 1.1 were mixed with the DMEM containing 10% serum to obtain the mixed culture medium containing the nanogels with different hardness, wherein the concentration of the nanogel was 200 μg/mL. Then, 2 mL of the mixed culture medium containing the nanogels with different hardness was added to each well of the 6-well plate containing M2 macrophages in step 1), incubated in an incubator for 24 hours, and the upper layer of the culture medium was aspirated. Then, 2 mL of the DMEM not containing serum was added to each well, incubated in an incubator at 37° C. and 5% CO₂ for 24 hours, the upper layer of the culture medium was aspirated, the cells were collected and washed 3 times with the PBS solution, the cells were digested with trypsin and then centrifuged, macrophages were collected, and the collected macrophages were mixed with 4T1 cells to obtain mixed cells. Macrophages collected after treatment by adopting the mixed culture medium containing 2% NGs nanogel were mixed with 4T1 cells according to a ratio of 2:1 as the 2% NGs treatment group. Macrophages collected after treatment by adopting the mixed culture medium containing 15% NGs nanogel were mixed with 4T1 cells according to a ratio of 2:1 as the 15% NGs treatment group. M2 macrophages prepared in step 1) were mixed with 4T1 cells according to a ratio of 2:1 as the M2 treatment group. M0 macrophages prepared in step 1) were mixed with 4T1 cells according to a ratio of 2:1 as an M0 treatment group. M1 macrophages prepared in step 1) were mixed with 4T1 cells according to a ratio of 2:1 as an M1 treatment group.

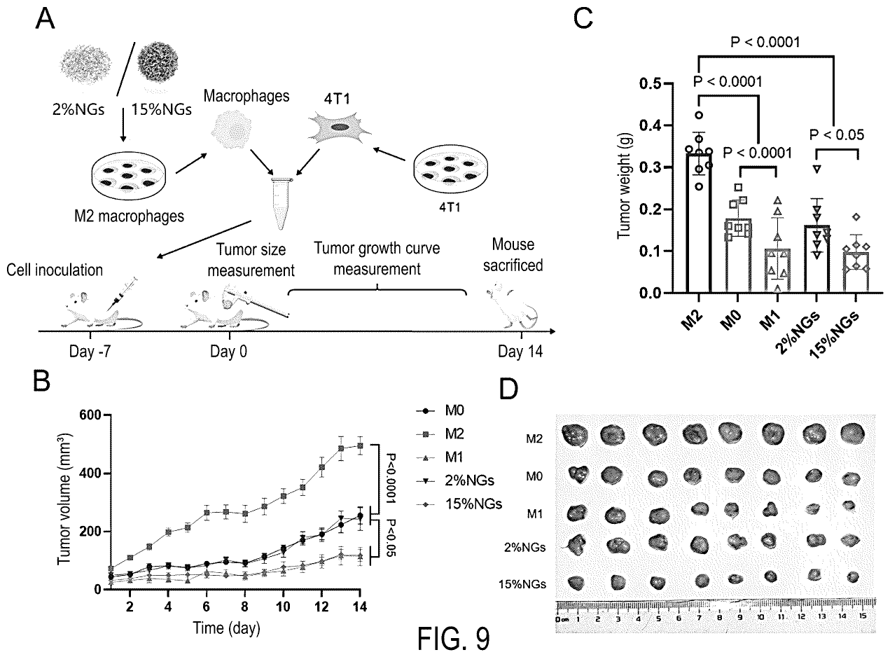

4) The mixed cells of the different treatment groups were subcutaneously inoculated on the back of female BALB/C mice near the right hind limb, the inoculation number was 3×10⁶, the inoculation volume was 100 μL, and a mixed model of a 4T1 breast cancer subcutaneous tumor of a mouse was constructed, as shown in content A of FIG. 9. When the tumor volume reached 100 mm³, day 1 was recorded, and a long side (a) and a short side (b) of the subcutaneous tumor of the mouse were measured using a vernier caliper every day, and the tumor volume was calculated according to the calculation formula: tumor volume V=(a×b²)/2. After the measurement on day 14, the mouse was sacrificed, and the tumor was removed, weighed, and photographed. The removed tumor was fixed with 4% paraformaldehyde and sliced, CD86 immunofluorescence was performed, and a CD86 area ratio was then quantified to evaluate the degree of repolarization of tumor-associated macrophages (TAMs).

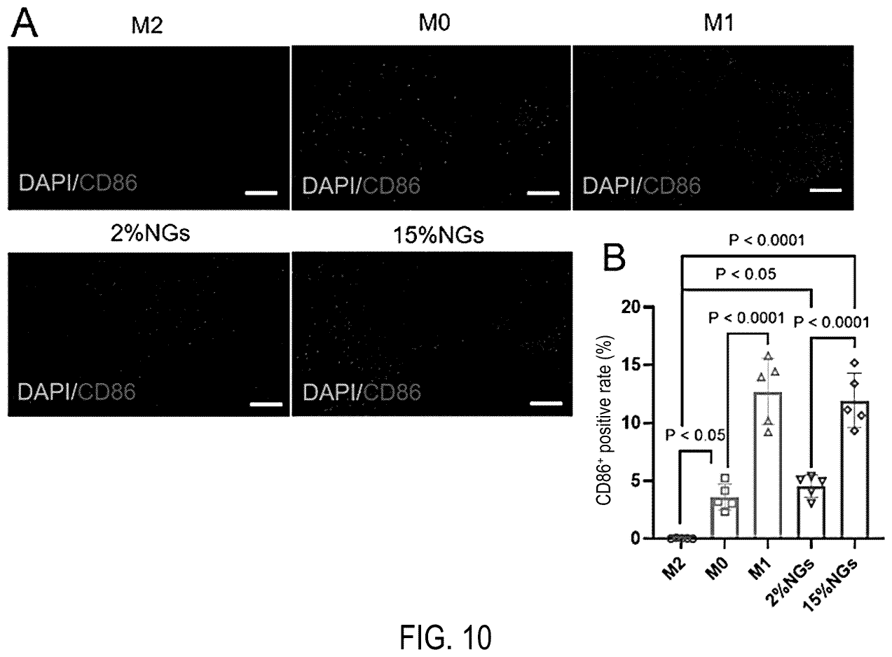

The results show that as can be seen from the tumor volume in content B of FIG. 9, the tumor weight in content C of FIG. 9, and the tumor photo in content D of FIG. 9, compared with the M2 treatment group, the volumes and the weights of the breast cancer tumors in the 2% NGs treatment group and the 15% NGs treatment group were significantly lower than those in the M2 treatment group. This indicates that after treating M2 macrophages using the nanogels with different hardness, tumor growth can be significantly affected, and the tumor killing effect is good. As can be seen from content A of FIG. 10 and content B of FIG. 10, compared with the M2 treatment group, the fluorescence intensities in the 2% NGs treatment group and the 15% NGs treatment group were enhanced, and the CD86⁺ positivity rates were significantly increased. This indicates that after treating M2 macrophages using the nanogels with different hardness, the expression of CD86⁺ can be significantly increased, improving the degree of polarization of M2 macrophages to M1 macrophages.

### Example 6 Inhibitory Effects of Nanogels on Mouse Tumor Growth

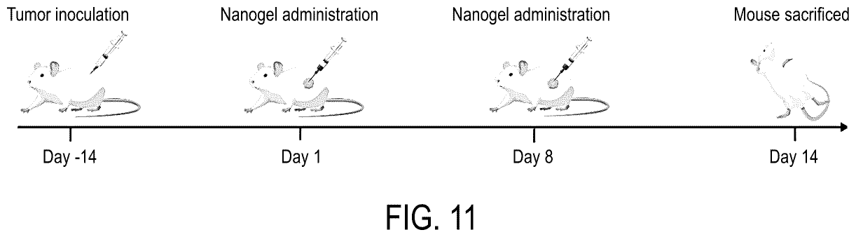

1×10⁶ 4T1 cells were subcutaneously inoculated on the back of female BALB/C mice near the right hind limb, the volume was 100 μL, and a model of a 4T1 breast cancer subcutaneous tumor of a mouse was constructed. When the tumor volume reached 200 mm³, day 0 was recorded, and mice were randomly divided into three groups, including the control group, the 2% NGs treatment group, and the 15% NGs treatment group. On day 1 and day 8 after grouping, the treatment groups were respectively intratumorally injected with 25 μL of normal saline, 2% NGs, and 15% NGs (the injection doses of the nanogels were all 20 mg/mL). The experimental process is shown in FIG. 11. Starting from day 1 after grouping, the long side (a) and the short side (b) of the subcutaneous tumor of the mouse were measured using a vernier caliper every day, and the tumor volume was calculated according to the calculation formula: tumor volume V=(a×b²)/2. After the measurement on day 14, the mouse was sacrificed.

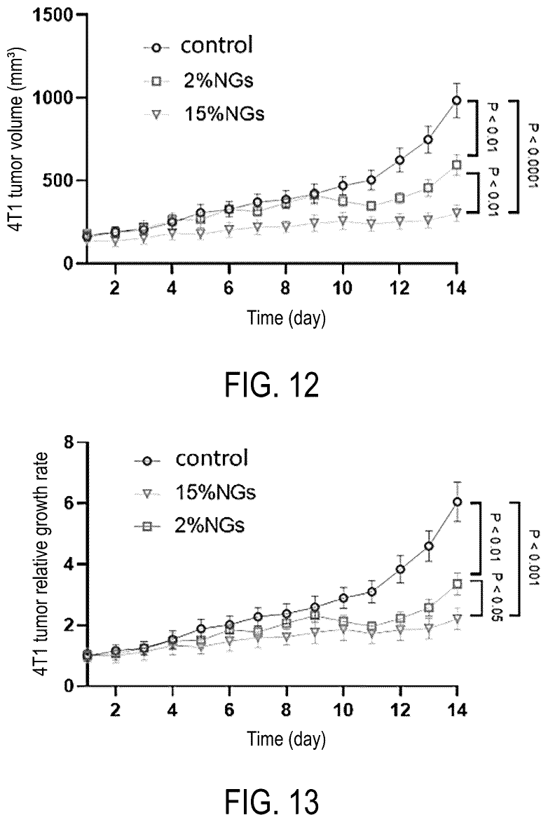

The results show that as can be seen from FIG. 12 and FIG. 13, the volume and the relative growth rate of the breast cancer tumors in the 2% NGs treatment group and the 15% NGs treatment group were significantly lower than those in the control group. This indicates that intratumoral injection of the nanogel with appropriate hardness can effectively inhibit tumor growth.

It is easy for persons skilled in the art to understand that the above is only a preferred embodiment of the disclosure and is not intended to limit the disclosure. Any modifications, equivalent substitutions, and improvements made within the spirit and the principle of the disclosure should be included in the protection scope of the disclosure.

What is claimed is:

1. An application of a nanogel in use or preparation of a drug for reverse polarization of M2 TAMs, wherein a Young's modulus of the nanogel is 20 to 600 KPa, and the nanogel reverse polarizes M2 TAMs to M1.

2. An application of a nanogel in use or preparation of an anti-tumor drug, wherein an active ingredient of the anti-tumor drug is the nanogel, a Young's modulus of the nanogel is 20 to 600 Kpa, and the nanogel reverse polarizes M2 TAMs to M1 when used for anti-tumor treatment, thereby inhibiting proliferation of tumor cells.

3. The application according to claim 1, wherein the Young's modulus of the nanogel is 50 to 600 KPa.

4. The application according to claim 1, wherein the nanogel is obtained by a polymerization reaction of a monomer initiated through an initiator in an aqueous phase under a condition of presence of a cross-linking agent and a surfactant,
the Young's modulus of the nanogel is regulated through regulating a molar ratio of the cross-linking agent to the monomer.

5. The application according to claim 4, wherein the monomer comprises one or more of a temperature-responsive monomer, a pH-responsive monomer, and a reduction-responsive monomer.

6. The application according to claim 5, wherein the temperature-responsive monomer is one or more of N-isopropylmethacrylamide, N-isopropylacrylamide, and N-ethylacrylamide; and/or
the pH-responsive monomer is one or more of methacrylic acid, acrylic acid, and 2-acrylamido-2-methyl-1-propanesulfonic acid; and/or
the cross-linking agent is one or more of N,N′-bis(acryloyl)cystamine, N,N′-methylenebisacrylamide, and N,N′-vinylbisacrylamide; and/or
the initiator is one or more of potassium persulfate, sodium persulfate, and tert-butyl hydroperoxide; and/or
the surfactant is one or more of sodium lauryl sulfate, sodium lauryl sulfonate, and lecithin.

7. The application according to claim 6, wherein the monomer comprises a temperature-responsive monomer and a pH-responsive monomer, a molar ratio of the pH-responsive monomer to the temperature-responsive monomer is (3˜8):100; and/or
a molar ratio of the cross-linking agent to the temperature-responsive monomer is (1˜20):100; and/or
a mass ratio of the initiator to the temperature-responsive monomer is (5˜15):550; and/or,
a mass ratio of the surfactant to the temperature-responsive monomer is (20˜35):550.

8. The application according to claim 4, wherein a reaction temperature of the polymerization reaction is 70° C. to 85° C., and a reaction time is 4 to 8 hours.

9. The application according to claim 2, wherein the tumor cells comprise one or more of liver cancer cells, breast cancer cells, colon cancer cells, lung cancer cells, esophageal squamous cell cancer cells, gastric cancer cells, ovarian cancer cells, prostate cancer cells, pancreatic cancer cells, lymphoma cells, melanoma cells, and glioblastoma cells.

10. The application according to claim 2, wherein the nanogel is administered through intratumoral injection when used for anti-tumor treatment,
preferably, a dosage of the nanogel is 1 to 10 μg/mm³.

11. The application according to claim 2, wherein the Young's modulus of the nanogel is 50 to 600 KPa.

12. The application according to claim 2, wherein the nanogel is obtained by a polymerization reaction of a monomer initiated through an initiator in an aqueous phase under a condition of presence of a cross-linking agent and a surfactant,
the Young's modulus of the nanogel is regulated through regulating a molar ratio of the cross-linking agent to the monomer.
